(19) United States
(12) Patent Application Publication
(10) Pub. No.: US 2022/0288289 A1
(43) Pub. Date: Sep. 15, 2022

(54) SMART MILK LETDOWN EXPRESSION SYSTEM

(71) Applicant: Olababy Incorporated, Beaverton, OR (US)

(72) Inventors: Qiao LIN, Beverly Hills, CA (US); Paul WANG, Portland, OR (US)

(21) Appl. No.: 17/692,152

(22) Filed: Mar. 10, 2022

Related U.S. Application Data
(60) Provisional application No. 63/160,767, filed on Mar. 13, 2021.

(51) Int. Cl. A61M 1/06; A61M 1/00; A61J 13/00

(52) U.S. Cl. CPC A61M 1/06; A61M 1/82; A61J 13/00; A61M 2205/075; A61M 2210/1007; A61M 2205/50; A61M 2205/3379

(57) ABSTRACT

A system includes a breast shield configured to mount to a breast during a breast milk letdown process. The breast shield includes one or more sensors integrally mounted thereon. The system also includes an analyzer configured to collect and analyze data obtained by the one or more sensors to extract information reflecting a condition of the breast.

### CROSS REFERENCE TO RELATED APPLICATION

This application claims the benefit of priority to U.S. Provisional Application No. 63/160,767, filed on Mar. 13, 2021. The content of the above-mentioned application is incorporated herein by reference in its entirety.

### TECHNICAL FIELD

The present disclosure relates generally to systems and devices and, more specifically, to a smart milk letdown expression system.

### BACKGROUND

Letdown refers to the natural reaction that happens during breast feeding or breast milk pumping. You may observe breast milk leaking or spraying out of the breast that the baby is not breastfeeding on. This can also happen to the breast that is not pumped while the other breast is being pumped. The letdown process can be slow, difficult, and painful. Difficulties with the letdown reflex can lead to blocked duct and low breast milk supply.

### SUMMARY OF THE DISCLOSURE

One aspect of the present disclosure provides a system including a breast shield configured to mount to a breast during a breast milk letdown process. The breast shield includes one or more sensors integrally mounted thereon. The system also includes an analyzer configured to collect and analyze data obtained by the one or more sensors to extract information reflecting a condition of the breast.

Other aspects of the present disclosure can be understood by those skilled in the art in light of the description, the claims, and the drawings of the present disclosure.

### BRIEF DESCRIPTION OF THE DRAWINGS

The following drawings are provided for illustrative purposes according to various disclosed embodiments and are not intended to limit the scope of the present disclosure.

FIG. 1 illustrates a schematic diagram of a smart letdown expression system, according to an embodiment of the present disclosure;

FIG. 2A illustrates a schematic front view of a breast milk letdown collecting device, according to an embodiment of the present disclosure;

FIG. 2B illustrates a schematic back view of the breast milk letdown collecting device shown in FIG. 2A, according to an embodiment of the present disclosure;

FIG. 2C illustrates a schematic side view of the breast milk letdown collecting device shown in FIG. 2A, according to an embodiment of the present disclosure; and

FIG. 3 illustrates a schematic front perspective view of the breast milk letdown collecting device coupled with a wearing strap, according to an embodiment of the present disclosure.

### DETAILED DESCRIPTION

Embodiments consistent with the present disclosure will be described with reference to the accompanying drawings, which are merely examples for illustrative purposes and are not intended to limit the scope of the present disclosure. Wherever possible, the same reference numbers are used throughout the drawings to refer to the same or similar parts, and a detailed description thereof may be omitted.

Further, in the present disclosure, the disclosed embodiments and the features of the disclosed embodiments may be combined. The described embodiments are some but not all of the embodiments of the present disclosure. Based on the disclosed embodiments, persons of ordinary skill in the art may derive other embodiments consistent with the present disclosure. For example, modifications, adaptations, substitutions, additions, or other variations may be made based on the disclosed embodiments. Such variations of the disclosed embodiments are still within the scope of the present disclosure. Accordingly, the present disclosure is not limited to the disclosed embodiments. Instead, the scope of the present disclosure is defined by the appended claims.

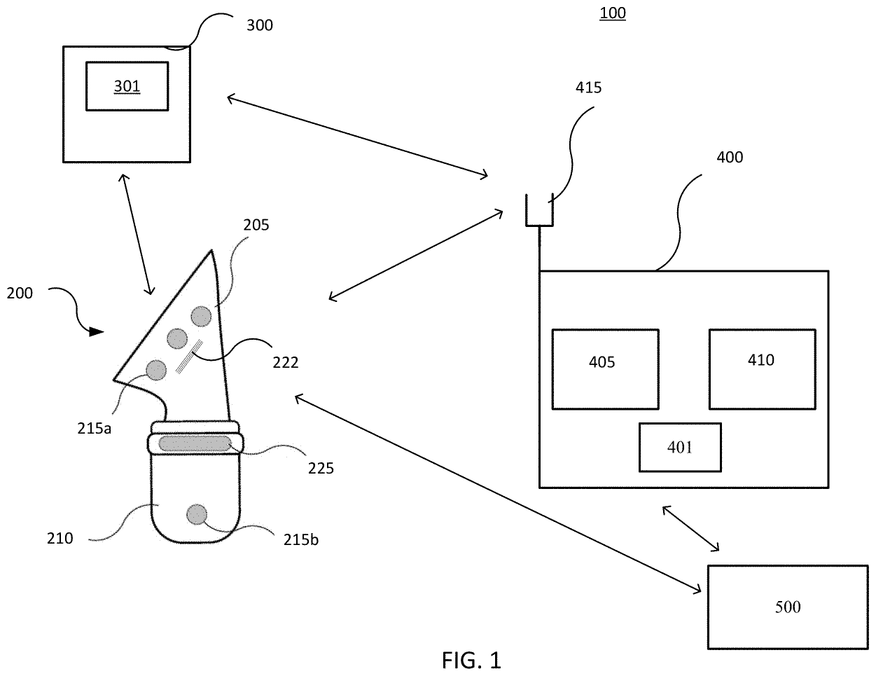

The disclosed smart breast milk letdown expression system is configured to collect letdown breast milk. FIG. 1 illustrates a smart breast milk letdown expression system 100 (also referred to as a system 100, or a smart breast milk letdown expression system 100), according to an embodiment of the present disclosure. As shown in FIG. 1, the smart breast milk letdown expression system 100 includes a breast milk letdown collecting device 200. In some embodiments, the smart breast milk letdown expression system 100 may also include a user terminal 300. In some embodiments, the smart breast milk letdown expression system 100 may also include an analyzer 400. In some embodiments, the smart breast milk letdown expression system 100 may also include a computing device 500 (e.g., a remote server). The user terminal 300, the analyzer 400, and the computing device 500 may include both hardware components (e.g., circuits, processors, memories, data storage devices, communication ports, antennas) and software components (e.g., computer codes, instructions, etc.).

The breast milk letdown collecting device 200 may include a breast shield 205 made of a flexible material such as silicone or rubber. The breast shield 205 may contact the breast during the operation. The breast milk letdown collecting device 200 may also include a container 210 (also referred to as a base, or receptacle). The container 210 may be a breast milk bottle. The container 210 may be coupled with the breast shield 205, and may receive the milk letdown produced by the breast. In some embodiments, the container 210 may be removably coupled with or attached to the breast shield 205. The container 210 may be detached from the breast shield 205 for cleaning, storage, etc. The container 210 may be coupled with or attached to the breast shield 205 through any suitable coupling mechanism, such as threads, snap-fit coupling, press-fit coupling, etc. The container 210 may be made of a flexible material, such as silicone, rubber, soft plastic, etc. In some embodiments, the material of the container 210 may be substantially the same as the material of the breast shield 205. For example, both of the breast shield 205 and the container 210 may be made of a silicone material. The container 210 may be squeezed by hand by the user. Squeezing the container 210 may produce a suction power, which may facilitate the breast milk letdown. Different containers may be used to couple with the breast shield 205 to generate different suction powers. A user can choose a container of a suitable size, shape, and/or material (which may have a specific softness) that can generate a comfortable suction power.

The breast milk letdown collecting device 200 may include one or more sensors (which may also be actuators or transducers) 215a, 215b disposed at various portions. It is noted that the term “sensor” used herein also encompasses actuators and transducers. For illustrative purposes, a plurality of sensors 215a, 215b are shown in FIG. 1. The sensors 215a may be built in the breast shield 205. The sensors 215a may be disposed at an inner surface, an outer surface, or within the body of the breast shield 205. The sensors 215a may be the same type of sensors, or may be different types of sensors. For example, the sensors 215a may include one or more pressure sensors, one or more stimulus signal generating sensors (which may also be referred to as stimulus actuators or transducers), one or more imaging sensors (e.g., thermo-imaging sensors, ultrasonic sensors, etc.), one or more thermal sensors (e.g., temperature sensors), and one or more fluid property measuring sensors (e.g., for measuring flow rate of the milk, the viscosity of the milk, etc.), one or more actuators or transducers for generating vibration, one or more transducers for generate heat or warmth, one or more transducers for generating pressure, etc. The various types of sensors 215a may be distributed at various portions of the breast shield 205. In some embodiments, one or more pressure sensors may be distributed at various parts of the breast shield 205 to measure contacting pressures between the breast shield 205 and the breast at various locations. In some embodiments, one or more temperature sensors may be distributed at various parts of the breast shield 205, which may be configured to measure temperatures at various locations of the breast. In some embodiments, one or more thermo-imaging sensors may be distributed at the breast shield 205, which may be configured to provide thermo-imaging data reflecting the inside structures of the breast. In some embodiments, a plurality of ultrasonic transducers (which may include emitters and receivers) may be distributed at the various parts of the breast shield 205. The ultrasonic transducers may be configured to provide ultrasonic signals reflecting the inside structures of the breast. In some embodiments, a plurality of piezoelectric transducers may be distributed at various parts of the breast shield 205. For example, one or more piezoelectric transducers disposed at one side of the breast may generate one or more wave signals propagating through the inside of the breast, and the wave signals may be received by one or more acoustic wave receivers (e.g., microphones) disposed at another side of the breast. The piezoelectric transducers and the acoustic wave receivers may together provide an acoustic signal indicating the inside structures of the breast. In some embodiments, the breast shield 205 may include one or more sensors configured to measure the flow rate of the milk production. In some embodiments, a flowrate measuring sensor may be installed at the lower, narrow part of the funnel shape (i.e., the neck part) of the breast shield 205 to measure the flow rate of the milk production. In some embodiments, the breast shield 205 may be made of a soft material, which may contract and expand. The actuators (e.g., piezoelectric actuators) included in the sensors 215a may cause breast shield 205 to contract and expand, which in turn may squeeze the breast at a suitable force. The contract of the breast shield 205 may also cause the total volume of the breast shield 205 and the container 210 to reduce, thereby creating a suction force. In some embodiments, the squeeze caused by the actuators provided on the breast shield 205 may also massage the breast.

In some embodiments, one or more sensors 215b may be embedded in one or more portions of the container 210 configured to contain the breast milk generated by the breast, such as the base, the wall, and/or the neck portions of the container 210. The sensors 215a and 215b distributed at the breast shield 205 and/or the container 210 may be powered by one or more power sources 225. The one or more power sources 225 may be rechargeable or non-rechargeable batteries. The one or more power sources 225 may be distributed at suitable locations of the breast shield 205 and/or the container 210. In one embodiment, a rechargeable battery may be disposed at a neck portion of the container 210 that is connected with the breast shield 205. The battery may be a button-type battery or any other suitable battery. In some embodiments, the power source 225 may be a wireless power receiver including an electric circuit and one or more electric components configured to receive an electric power transmitted wirelessly to the power source 225. For example, the electric power may be transmitted to the power source 225 disposed at a location between the breast shield 205 and the container 210 from the analyzer 400. Any suitable non-contact, wireless power transmission technology may be used, such as near field transmission.

In some embodiments, at least one of the sensors 215b may be different from at least one of the sensors 215a. In some embodiments, at least one of the sensors 215b may be the same as at least one of the sensors 215a. In some embodiments, all of the sensors 215b are of the same type of sensors. When disposed at the inner surface of the breast shield 205, the sensors 215a may be in direct contact with a breast when the breast shield 205 is placed over the breast. In some embodiments, the sensors 215b may include a volume sensor configured to measure a volume of the collected milk.

The analyzer 400 may be configured to communicate with the sensors 215a and 215b through wired or wireless communication to collect data, signals, and/or information acquired by the sensors. In some embodiments, the analyzer 400 may also provide control commands to the sensors 215a and 215b to control the operations of the sensors. In some embodiments, the analyzer 400 may be a standalone device separate from the breast shield 205 and the container 210. In some embodiments, the analyzer 400 may be an integral part of the breast shield 205 and/or the container 210. For example, the analyzer 400 may be a standalone device configured for collecting signals, data from the sensors 215a and 215b that are disposed at the breast shield 205 and/or the container 210. In some embodiments, the analyzer 400 may be an integral part of the user terminal 300 (e.g., the analyzer may include software and/or hardware components of the user terminal 300). In some embodiments, the analyzer 400 may process (e.g., analyze) the collected sensor data and display analysis results to a user or transmit the analysis results to another device (e.g., a computing device 500, such as a remote server or a local computer, and/or the user terminal 300). In some embodiments, the analyzer 400 may be a data transceiver including both hardware components (e.g., circuits, logic gates, etc.) and software components (e.g., codes, instructions). For example, the analyzer 400 may collect data from the sensors 215a and 215b, and may transmit the collected data to the computing device 500 and/or the user terminal 300. The user terminal 300 may be a smart phone, a tablet, a laptop, a desktop, a personal digital assistant, etc. In some embodiments, the analyzer 400 may not process the data, and may simply relay the collected raw sensor data to another device, such as the computing device 500 and/or the user terminal 300. Thus, the term “analyzer” is for convenience, and does not necessarily indicate that the analyzer performs data processing of the collected sensor data. Although FIG. 1 shows the analyzer 400 being separated from the user terminal 300 and the computing device 500, it is understood that in some embodiments, the analyzer 400 may be part of the user terminal 300 and/or the computing device 500. In some embodiments, the sensors 215a and 215b disposed on the breast shield 205 and/or the container 210 may communicate (e.g., transmit sensor data) to the analyzer 400 through a wired or wireless communication. In some embodiments, the sensors 215a and 215b disposed on the breast shield 205 and/or the container 210 may communicate (e.g., transmit sensor data) to the computing device 500 (e.g., a remote server or a local computer) through a wired or wireless communication. In some embodiments, the sensors 215a and 215b disposed on the breast shield 205 and/or the container 210 may communicate (e.g., transmit sensor data) to the user terminal 300 through a wired or wireless communication. Although not shown, in some embodiments, the analyzer 400 may include a display screen, such as a touch screen, for receiving user input and/or displaying data to the user. As shown in FIG. 1, in some embodiments, the analyzer 400 may communicate with the user terminal 300 and/or the computing device 500 to transmit and/or receive data through a communication device 415. The communication device 415 may include a communication interface or port, and/or a communication antenna. In some embodiments, the analyzer 400 may include a remote controller function for controlling the on/off of the sensors 215a and 215b, for selecting a pre-programmed stimulus signal to be generated by one or more sensors 215a (or for selecting a pre-programmed massage routine for massaging the breast). The remote control function may include any other suitable functions for a user to select to control the operations of the sensors. The user may control the sensors 215a, 215b, and the power source 225 through a user interface 301 and/or 401 provided at the user terminal 300 and/or the analyzer 400. In some embodiments, the user may provide user input at the user interface to control the on and off status of the sensors by controlling the power source. In some embodiments, the user may control which data to collect by the sensors. In some embodiments, the user may control when to collect the data through the sensors. In some embodiments, the user may control which massaging routine/program to apply to the breast.

The smart breast milk letdown expression system 100 may collect the data measured by the various sensors 215a and 215b (i.e., sensor data). For example, various data provided by various sensors 215a and 215b may be stored in a data storage device 410 (e.g., a hard drive, a memory, a USB drive, etc.) of the analyzer 400. The data storage device 410 may also be referred to as a non-transitory computer-readable medium. The analyzer 400 may include a processor 405 configured to process the data and extract information reflecting the health condition of the breast. The processor 405 may be, e.g., a central processing unit, a graphics processing unit, or any other processor having computing capabilities, and may include both hardware components (e.g., circuits, gates, etc.) and software components (e.g., computer codes, instructions). For example, the information may include data reflecting the amount of milk produced, data reflecting a prediction of milk to be produced data reflecting anomaly in the amount of milk production, anomaly relating to the general health of the breast (such as a change in the inside structure of the breast). The change in the inside structure of the breast may be a change that may affect the production of the milk, or a change that may affect the health condition of the breast.

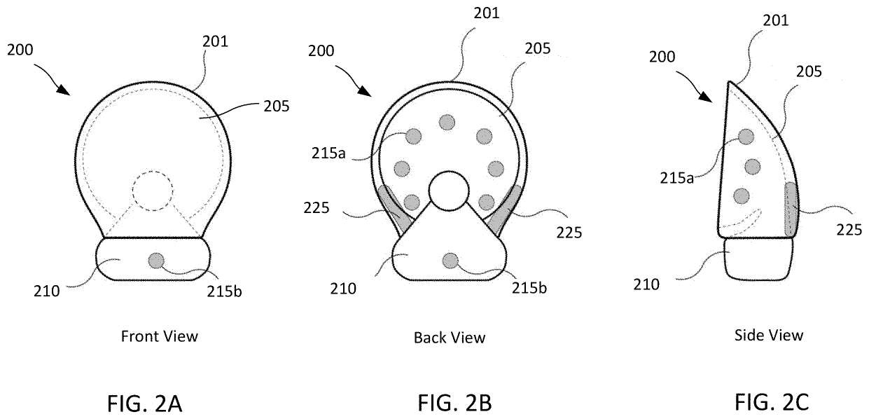

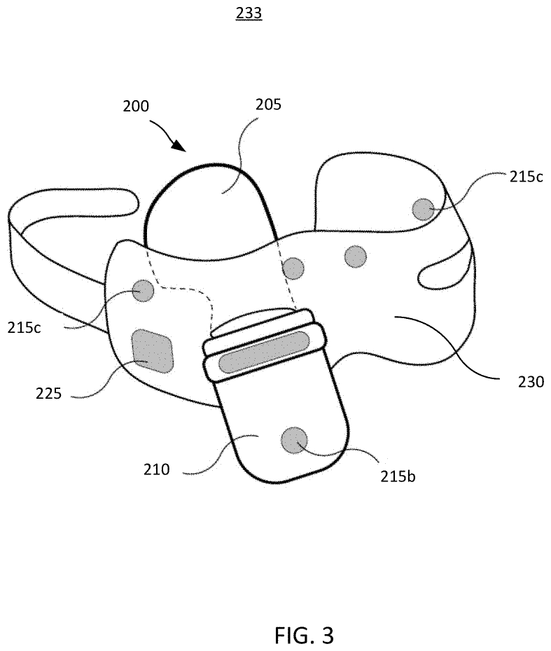

In some embodiments, the sensors 215a may include a plurality of stimulus signal generating sensors distributed at various parts of the breast shield 205. In some embodiments, the power source 225 may provide an electric power to the sensors 215a for the normal operations of the sensors. For example, one or more sensors 215a may be actuators configured to generate one or more stimulus signals when a suitable power is provided to the actuators. The stimulus signals may provide a stimulus to the breast through the actuators and/or the breast shield 205. The stimulus signals may be vibration signals, pressure signals, and/or heat signals. The stimulus signals may apply massage to the breast directly or through the breast shield 205, which may facilitate the production of the milk (e.g., increase the milk production, reduce the soreness of the breast, reduce the chance of clogging of the milk duct inside the breast, etc.). For example, the sensors 215a may include one or more piezoelectric actuators or transducers mounted on the breast shield 205. When a power is provided to a piezoelectric actuator, a vibration may be produced by the piezoelectric actuator. The piezoelectric actuators may cause a portion of or the entire breast shield 205 to vibrate, thereby massaging the breast. Different vibration patterns may be generated by supplying different power patterns. In some embodiments, one or more heating elements 222 (e.g., wires or pads, which may also be referred to as thermo-transducers) may be embedded in the breast shield 205. When an electric power is provided to the heating elements 222, heat may be generated to warm the breast shield 205. In turn, the breast shield 205 may warm the breast, which may reduce clog and facilitate production of milk. In some embodiments, the heating elements 222 may directly contact and heat the breast. In some embodiments, the stimulus signals may be generated during letdown collection/pumping or may be generated during any time selected by the user. In some embodiments, the smart breast milk letdown expression system 100 may include a plurality of pre-programmed routines for the user to select for massaging the breast. It is noted that the heating elements 222 may also be included in the embodiments shown in FIGS. 2A-2C and FIG. 3, although for the heating elements 222 are not shown in these figures. The smart breast milk letdown expression system 100 may provide recommendations for the user based on the data collected through the various sensors. Such programs may be installed in the analyzer 400 or in a smart device (e.g., user terminal 300), such as a phone, tablet, or a computer of the user. In some embodiments, the analyzer 400 may also function as a remote controller for a user to select the programmed routine. The analyzer 400 may provide selection buttons, keys, or a user interface (e.g., a graphic user interface) for the user to select a routine and/or to control the operations of the sensors, actuators, and/or transducers (collectively referred to as 215a and 215b). The analyzer 400 may also enable the user to share the measured sensor data to healthcare workers, facilities, and/or communities, such as online social media, friends, family members.

In some embodiments, the data collected by the smart breast milk letdown expression system 100 can be summarized, analyzed, and shared with the users via a mobile application (or “APP”), which may be installed on the user terminal 300, such as a smart phone, a tablet, a computer, etc. In some embodiments, the APP may be installed on the analyzer 400. In some embodiments, the analyzer 400 may be the same as the user terminal 300, or may include the user terminal 300, or may be part of the user terminal 300. In some embodiments, the analyzer 400 may be separate from the user terminal 300. For example, the analyzer 400 may collect data from the sensors 215a and 215b and analyze the data, and may transmit the analysis results to the user terminal 300 for display to the user. In some embodiments, the analyzer 400 may collect the data from the sensors 215a and 215b, and transmit the data (which may have been pre-processed to remove noise, etc.) to the user terminal 300, and the APP installed on the user terminal 300 may analyze the data and present the analysis results to the user.

To protect the user privacy, in some embodiments, the smart breast milk letdown expression system 100 may include a standalone analyzer 400 that collects data, analyzes data, and presents results to the user. In some embodiments, the analyzer 400 may include a display screen, such as a touch screen. In some embodiments, the analyzer 400 may not include a display screen, and may transmit data to other devices, such as a separate display or the user terminal for displaying data. Analysis results may be displayed to the user on the user terminal 300 or on the analyzer 400.

In some embodiments, upon user consent, the analyzer 400 may transmit data to the computing device 500 (e.g., a remote server, such as a cloud-based computing device) through a wireless communication channel, such as WiFi, Bluetooth, cellular channel, or through a wired communication channel, such as Ethernet. In some embodiments, the analyzer 400 may be part of the computing device 500. The computing device 500 may include any suitable hardware components and/or software components for facilitating communication with the other devices included in the system 100, for obtaining data from the other devices, for controlling the other devices, and/or for performing computations based on data received from the other devices. The hardware components may include a processor, a memory (or data storage device), a communication antenna or port, a display (or a user interface), etc. The computing device 500 (e.g., remote server) may be a part of the smart breast milk letdown expression system 100, or may be a third party secured server. In some embodiments, the sensors 215a provided on the breast shield 205 and/or the sensors 215b on the container 210 may directly communicate with the computing device 500 (e.g., remote server). The computing device 500 (e.g., remote server) may collect data from a plurality of users within a community, within a medical facility, within an organization, within a county, within a state, within a country, or within several countries. The data from the plurality of users may be analyzed by the computing device 500 using any suitable big data analysis tools, such as machine learning and/or artificial intelligence tools. The analysis results from various users may benefit individual users as well. For example, data from one user may be compared with statistical analysis of data from various users to determine whether any data of the individual user significantly deviate from a normal or typical range obtained from the statistic results of the data of other users. The deviation may indicate an anomaly inside the structure of the breast or a degradation of the general health condition of the breast of the specific user. For example, the sensors 215a and/or 215b may collect information relating to the temperature of the breast, the pressure of the breast, the flow rate of the milk, and the volume of the milk during each breast milk letdown process. The sensed information may be provided to the analyzer 400 along with time information (e.g., when the letdown is performed). The analyzer 400 may preliminarily analyze the sensed information, compare the sensed information received from the analyzer 400 with historical information of the same user that is stored in the data storage device 410, and optionally compare the sensed information with statistical data obtained from analyzing sensed information of other users. The analyzer 400 may provide recommendations to the user through the user terminal 300. For example, the analyzer 400 may recommend the user to massage the breast before, during, or after the letdown process, and may display the recommendation and/or a recommended massaging routine/program to the user on a display screen of the user terminal 300. The massaging routine/program may correspond to specific amplitudes of voltages to be applied to the actuators included in the sensors 215a for causing vibration to the breast, and specific amplitudes of voltages to be applied to the heating elements 222 included in the sensors 215a for heating the breast. In some embodiments, the above-described data analysis may be at least partially or entirely performed by the computing device 500. In some embodiments, the data analysis may be at least partially performed by the user terminal 300.

In some embodiments, users of the smart breast milk letdown expression system 100 (e.g., through the APP) may form online social media communities, such as breastfeeding mothers' association. Information relating to the breastfeeding, as well as personalized recommendations provided by the smart breast milk letdown expression system 100 to the individual users may be shared within the social media communities in a voluntary basis. Such information sharing may benefit various users who are experiencing issues with producing milk.

In some embodiments, various sensors 215a and 215b may be built into the breast shield 205 and/or the container 210. The breast shield 205 and/or the container 210 may be made of a flexible material, such as silicone. The sensors 215a and 215b may be integrated into the silicone (hence sealed inside) and may be isolated from water or moisture, which may damage the sensors 215a and 215b. The sensors 215a and 215b may communicate with the analyzer 400 wirelessly through WiFi, Bluetooth, or any other wireless communication channels. The sensors 215a and 215b may communicate with the communication device 415 of the analyzer 400. The sensors 215a and 215b may provide sensed data relating to the breast and/or the milk letdown to the analyzer 400. In some embodiments, the sensors 215a and 215b may directly communicate with the user terminal 300 through WiFi or Bluetooth, and may transmit data to the user terminal 300 instead of the analyzer 400. The flexible nature of the silicone container 210 will generate a natural suction force when squeezed and released onto the breast. A user can control the amount of suction force by interchanging to a container of a different size and/or material. In some embodiments, the power source 225 (e.g., battery) may be integrated with a portion of the breast shield 205 to provide power to the various sensors 215a, or each sensor 215a or 215b may have an integrated power source (such as a battery).

Users can feed their babies directly from the container 210 or store breast milk collected in the container 210 directly in a freezer by replacing the breast shield 205 with other accessories such as a nipple or travel cap. The breast shield 205 has at least one surface (e.g., the inner contacting surface) that is made of a flexible material, such as silicone. In some embodiments, the sensors 215a may include one or more actuators to produce vibration, and one or more heating elements 222 to provide heating to the breast. The vibration can massage the breast. The heating can keep the breast at a comfortable temperature. The massage and the heating may stimulate the breast and induce better letdown experience. In some embodiments, the container 210 may be filled with warm water. The user can submerge the nipple in the warm water while the breast is massaged and the glands are suctioned to remove potential clogs.

FIGS. 2A-2C show various views (front, back, and side) of an integral design with the breast shield 205 integrated with the container 210 as a single piece. That is, the container 210 may not be removable from the breast shield 210. The integral design may include an outer shell 201. The breast shield 205 may be disposed inside the outer shell 201. The sensors 215a may be integrated with the breast shield 205. The sensors 215b may be integrated with the container 210. In some embodiments, two or more power sources (e.g., batteries) may be provided on each side of the breast shield 205. In some embodiments, the integral piece including the breast shield 205 and the container 210 may be made of silicone.

FIG. 3 shows a letdown assembly 233 including the breast milk letdown collecting device 200 coupled with a wearing strap 230, according to an embodiment of the present disclosure. The wearing strap 230 may be made of one or more suitable materials. For example, a portion of the wearing strap 230 may be made of a flexible material, such as cloth, silicone, etc. The wearing strap 230 may include a hole. In some embodiments, the neck (narrow) portion of the breast shield 205 may be located in the hole. In some embodiments, the top portion of the container 210 may be located in the hole. The free ends of the wearing strap 230 may be fastened together through any suitable mechanism. The wearing strap 230 may help secure the attachment of the breast shield 205 to the breast during a letdown process. In some embodiments, one or more sensors 215c of suitable types (including actuators, and/or transducers, which may be similar to the sensors 215a and/or 215b) may be integrated with the wearing strap 230. The sensors 215c may acquire data (e.g., temperature, pressure, acoustic signals, imaging signals, etc.) relating to the breast, as described above. For example, the sensors 215c may measure the temperature or pressure of the breast. In some embodiments, the sensors 215c may also measure the heart beats of the heart. In some embodiments, the wearing strap 230 may include a power source 225 (e.g., a battery) to provide power to the sensors 215c (which may include actuators, and/or transducers). In some embodiments, the sensors 215c may include actuators and/or transducers (e.g., piezoelectric actuators) configured to provide the stimulus signals to massage the body of the user, as described above. The wearing strap 230 may be used in combination with the breast milk letdown collecting device 200 of the one-piece design shown in FIGS. 2A-2C, or of the two-piece design shown in FIG. 1. It is noted that the sensors 215a disposed on the breast shield 205 are not shown for the simplicity of illustration.

The present disclosure provides a smart milk letdown expression system which can provide data reflecting or indicating the health condition of the breast, provide massage to the breast to reduce soreness, reduce clog, improve milk production, and provide an early sign of any potential issue in the breast. The scope of the present disclosure is defined in the appended claims.

What is claimed is:

1. A system, comprising:
a breast shield configured to mount to a breast during a breast milk letdown process, the breast shield including one or more sensors integrally mounted thereon; and
an analyzer configured to collect and analyze data obtained by the one or more sensors to extract information reflecting a condition of the breast.

2. The system of claim 1, wherein the breast shield includes a silicone material.

3. The system of claim 1, further comprising a container coupled with the breast shield and configured to receive milk produced by the breast.

4. The system of claim 3, further comprising one or more sensors integrally disposed at the container and configured to obtain data relating to the collected milk.

5. The system of claim 3, wherein the breast shield and container are detachably connected.

6. The system of claim 3, wherein the breast shield and the container are integrally formed as a single piece based on a same material.

7. The system of claim 3, wherein the container includes a silicone material.

8. The system of claim 3, wherein the breast shield and the container are substantially made of a same silicone material.

9. The system of claim 3, further comprising a power source provided at the breast shield or the container.

10. The system of claim 9, wherein the power source is a battery.

11. The system of claim 1, wherein the analyzer is configured to provide recommendations for massaging the breast to a user of the system.

12. The system of claim 1, further comprising a wearing strap coupled with the breast shield.

13. The system of claim 12, wherein the wearing strap includes one or more sensors configured to measure at least one of a temperature or a pressure of the breast.

14. The system of claim 3, further comprising a wearing strap coupled with the breast shield or the container.

15. The system of claim 14, wherein the wearing strap includes one or more sensors configured to measure at least one of a temperature or a pressure of the breast.

16. The system of claim 14, wherein the wearing strap includes one or more actuators configured to provide vibration to massage a body of a user of the system.

17. The system of claim 1, wherein the breast shield includes one or more actuators configured to provide vibration to massage the breast.

18. The system of claim 1, wherein the breast shield includes one or more heating elements configured to provide heat to sooth the breast.

19. The system of claim 1, further comprising a user terminal wirelessly connected with the sensors, and configured to provide a user interface for receiving a user input for controlling the sensors.

20. The system of claim 1, further comprising a remote server configured to provide recommendations relating to milk letdown to a user of the system based on information collected from other users of similar systems.
